(19) United States
(12) Patent Application Publication
(10) Pub. No.: US 2024/0226569 A9
(43) Pub. Date: Jul. 11, 2024

(54) HYPOGLOSSAL NERVE STIMULATION TO TREAT OBSTRUCTIVE SLEEP APNEA IN THE ABSENCE OF A RESPIRATORY SIGNAL

(71) Applicant: The Alfred E. Mann Foundation for Scientific Research, Valencia, CA (US)

(72) Inventors: Brian MECH, Buffalo, MN (US); Brian M. Shelton, Altadena, CA (US); Neil H. Talbot, La Crescenta, CA (US)

(21) Appl. No.: 18/489,403

(22) Filed: Oct. 18, 2023

Related U.S. Application Data
(60) Provisional application No. 63/417,324, filed on Oct. 19, 2022.

(51) Int. Cl. A61N 1/36

(52) U.S. Cl. CPC A61N 1/36139; A61N 1/3606

(57) ABSTRACT

The disclosure provides systems and methods for treating sleep-disordered breathing (e.g., obstructive sleep apnea) using a system comprising an inertial measurement unit (IMU) which includes an accelerometer and/or a gyroscope, wherein the IMU is configured to detect movement by a human subject and to generate data based on the detected movement. Positional and/or movement data generated by the IMU is used by a stimulation system to detect gross movement by the subject, and to deliver electrical stimulation to a nerve which innervates an upper airway muscle (e.g., a hypoglossal nerve) in response to the detection of gross movement, in order to treat the subject.

### CROSS-REFERENCE TO RELATED APPLICATION

This application claims the benefit of U.S. Provisional Application No. 63/417,324, which was filed on Oct. 19, 2022, and is expressly incorporated by reference herein in its entirety.

### BACKGROUND

Obstructive Sleep Apnea (“OSA”) is a sleep disorder involving obstruction of the upper airway during sleep. The obstruction of the upper airway may be caused by the collapse of or increase in the resistance of the pharyngeal airway, often resulting from tongue obstruction. The obstruction of the upper airway may be caused by reduced genioglossus muscle activity during the deeper states of NREM sleep. Obstruction of the upper airway may cause breathing to pause during sleep. Cessation of breathing may cause a decrease in the blood oxygen saturation level, which may eventually be corrected when the person wakes up and resumes breathing. The long-term effects of OSA include high blood pressure, heart failure, strokes, diabetes, headaches, and general daytime sleepiness and memory loss, among other symptoms.

OSA is extremely common and may have a prevalence similar to diabetes or asthma. Over 100 million people worldwide suffer from OSA, with about 25% of those people being treated. Continuous Positive Airway Pressure (CPAP) is a conventional therapy for people who suffer from OSA. More than five million patients own a CPAP machine in North America, but many do not comply with use of these machines because they cover the mouth and nose and, hence, are cumbersome and uncomfortable.

Neurostimulators may be used to open the upper airway as a treatment for alleviating apneic events. Such therapy may involve stimulating the nerve fascicles of the hypoglossal nerve (HGN) that innervate the intrinsic and extrinsic muscles of the tongue in a manner that prevents retraction of the tongue which would otherwise close the upper airway during the inspiration period of the respiratory cycle.

The rationale for this treatment method appears to be that it is enough simply to tone the tongue muscle and other nearby muscles, so that the tongue muscle does not retract in a manner that would cause OSA. The belief is that it is not necessary to specifically target the protraction (i.e., anterior movement) of the tongue muscle and to synchronize the occurrence of tongue protraction when it is most needed, i.e., during inspiration.

While it is possible to perform hypoglossal nerve stimulation (HGNS) in the absence of a respiration signal and still achieve clinically meaningful outcomes, improved outcomes are likely when stimulation is synchronized with the respiration cycle of a subject being treated. Prior systems have attempted to address this issue by integrating an inertial measurement unit (“IMU”) within the implantable pulse generator (“IPG”) used to apply electrical stimulation to one or more nerves of a subject being treated, in order to obtain a respiration signal. One significant challenge with this approach is that a respiration signal is small in comparison to those associated with gross body movements, making detection of a respiratory signal challenging or even impossible. Individuals with sleep-disordered breathing conditions (e.g., OSA) often move at least several dozen times per night. Thus, there remains a need for improved systems and methods that are capable of effectively treating sleep-disordered breathing conditions such as OSA, particularly in the absence of a respiratory signal. There further exists a need for systems and methods which can provide increased accuracy with respect to the detection of a subject's respiratory cycle and leveraging this increased accuracy to better treat subjects suffering from OSA.

### BRIEF SUMMARY OF EXEMPLARY ASPECTS OF THE DISCLOSURE

In an effort to synchronize stimulation with the respiratory cycle, current systems typically include an IMU within the IPG implanted in the subject being treated, in order to obtain a respiration signal. However, as noted above this approach is challenging because respiratory signals are weak in comparison to those associated with gross body movements. Individuals suffering from OSA and other sleep-disordered breathing conditions will often experience hundreds of gross movements during the course of a night (e.g., each apnea or hypopnea event will often result in a body movement). Thus, implanted IMUS known in the art are often unable to reliably detect the respiration signal of a subject and to achieve synchronization between stimulation and the respiratory cycle.

The present disclosure is directed to systems and methods that address these and other shortcomings of prior treatment systems. For example, in some aspects, the disclosure provides systems that utilize an IMU configured to detect gross movement instead of a respiration signal, based on the assumption that, for the purposes of HGNS, it is safe to assume that every time there is a gross movement an apnea-hypopnea index (“AHI”) event is occurring. In such situations, stimulation should begin immediately, or after some fixed amount of time, to curb the event(s), despite the fact that there is no discernable respiratory signal. In doing so, the IMU serves as a proxy for a respiration sensor, and also functions as an accurate AHI detector. Systems based on this approach may require fewer sensors, resulting in a simpler stimulation system that is likely to be more convenient for the subject and which may present further benefits (e.g., reduced processing and power demands due to the limited sensor requirements).

The following presents a simplified summary of several exemplary embodiments in order to provide a basic understanding of the inventions described herein. This summary is not intended as an extensive overview of all contemplated aspects and is intended to neither identify key or critical elements of all aspects nor delineate the scope of any or all aspects. Its sole purpose is to present some concepts of one or more aspects in a simplified form as a prelude to the more detailed description that is presented later.

In a first general aspect, the disclosure provides a system for treating a respiratory disorder, comprising: an IMU comprising an accelerometer and/or a gyroscope, wherein the IMU is configured to detect movement by a human subject, and to generate data based on the detected movement; and a stimulator comprising: a stimulation system configured to deliver stimulation to a nerve which innervates an upper airway muscle; and a controller coupled to the stimulation system, and to the IMU; wherein the controller is configured to detect gross movement by the human subject based on the data generated by the IMU, and to cause the stimulation system to stimulate the nerve in response to the detection of the gross movement.

In some aspects, the controller is further configured to detect the gross movement based on at least one predetermined threshold.

In some aspects, the at least one predetermined threshold comprises: a) a frequency of movements by the human subject; b) a distance of movement by the human subject; c) a velocity and/or acceleration of one or more movements by the human subject; d) a degree of rotational movement by the human subject; e) a magnitude of one or more accelerations, translational or angular, by the human subject; f) a magnitude of one or more velocities, translational or angular, by the human subject; g) an angular acceleration, velocity, and/or number of gross movement events per hour detected by the controller; and/or h) a threshold level of one or more frequencies in a time domain analysis of the IMU data.

In some aspects, the controller is further configured to detect the gross movement based on at least one predetermined threshold, and the at least one predetermined threshold comprises one or more of: a) a detection of at least 1, 2, 3, 4, 5, 6, 7, 8, 9, or 10 movements by the human subject within 15, 30, 45, 60, 75, or 90 seconds; b) a detection of the human subject, or a portion thereof, moving a distance of at least 5, 6, 7, 8, 9, 10, 11, 12, 13, 14, or 15 cm; c) a velocity of one or more movements by the human subject exceeding 0.1, 0.2, 0.3, 0.4, or 0.5 m/s; d) a degree of rotational movement by the human subject exceeding 1, 2, 3, 4, 5, 6, 7, 8, 9, or 10°; e) a magnitude of one or more accelerations, translational or angular, by the human subject exceeding 0.1, 0.2, 0.3, 0.4, or 0.5 m/s² and/or 90°/s; and/or f) a threshold level of one or more frequencies in a time domain analysis of the data generated by the IMU, wherein fₓ/f₀>5%, where fₓ is a signal strength at a predetermined frequency, optionally wherein the predetermined frequency is 0.25 hz.

In some aspects, the controller is further configured to cause the stimulation system to stimulate the nerve: a) immediately upon detection of the gross movement; or b) after a predetermined amount of time following the detection of the gross movement.

In some aspects, the controller is further configured to cause the stimulation system to stimulate the nerve: a) for a predetermined amount of time; b) until the data generated by the IMU indicates that the human subject has a normal respiratory signal; or c) until the data generated by the IMU indicates that the human subject has stopped gross movements.

In some aspects, the stimulation system is configured to deliver stimulation to a hypoglossal nerve and/or one or more branches of the hypoglossal nerve, and/or an ansa cervicalis, and/or a vagus nerve, and/or a phrenic nerve of the human subject.

In some aspects, the stimulation system is configured to deliver stimulation to a hypoglossal nerve and/or one or more branches of the hypoglossal nerve, and/or an ansa cervicalis, and/or a vagus nerve, and/or a phrenic nerve of the human subject.

In some aspects, the IMU is further configured to detect chest and/or abdominal movement by the subject; and the controller is further configured to detect a respiratory signal of the human subject based on the detected chest and/or abdominal movement; and to cause the stimulation system to cease stimulating the nerve or to refrain from stimulating the nerve, when the respiratory signal indicates that human subject is not experiencing an apnea or hypopnea event.

In some aspects, the system further comprises a heart rate sensor; and the controller is further configured to detect a heart rate of the human subject, and to take the human subject's heart rate into account when selecting a start or endpoint time, an intensity level, and/or a duration parameter for stimulation of the nerve.

In some aspects, the system is further configured to receive input from the human subject allowing a clinician or the human subject to control one or more parameters of the stimulation system.

In some aspects, the controller is further configured to allow the clinician or the human subject to place the controller in a standby mode in which the controller ceases to detect gross movement by the human subject and/or cause stimulation of the nerve.

In some aspects, the gross movement comprises a heaving motion of a chest and/or abdomen of the human subject, and the controller is further configured to detect the heaving motion based on a velocity, and/or an acceleration, and/or a direction, and/or a frequency of the heaving motion calculated based on the data generated by the IMU.

In some aspects, the gross movement comprises a heaving motion, and the controller is further configured to detect the heaving motion based on a magnitude, direction, and/or frequency of the heaving motion calculated using the data generated by the IMU.

In some aspects, the controller is further configured to cause stimulation of the nerve based on a frequency of the heaving motions; optionally, after detecting 1, 2, 3, 4, or 5 heaving motions within a predetermined time period.

In a second general aspect, the disclosure provides a method for treating sleep-disordered breathing using any of the systems described herein. In some aspects, a method according to the disclosure may comprise: detecting movement by a human subject using an inertial measurement unit (IMU) comprising an accelerometer and/or a gyroscope; deriving positional and/or movement data based on the IMU data; detecting, by a controller communicatively linked with the IMU, gross movement by the human subject based on the positional and/or movement data generated by the IMU; and stimulating a nerve innervating an upper airway muscle of the human subject in response to the detection of the gross movement.

In some aspects of the methods described herein, the controller is further configured to detect the gross movement based on at least one predetermined threshold.

In some aspects of the methods described herein, the at least one predetermined threshold comprises: a) a frequency of movements by the human subject; b) a distance of movement by the human subject; c) a velocity and/or acceleration of one or more movements by the human subject; d) a degree of rotational movement by the human subject; e) a magnitude of one or more accelerations, translational or angular, by the human subject; f) a magnitude of one or more velocities, translational or angular, by the human subject; g) an angular acceleration, velocity, and/or number of gross movement events per hour detected by the controller; and/or h) a threshold level of one or more frequencies in a time domain analysis of the IMU data.

In some aspects of the methods described herein, the controller is further configured to detect the gross movement based on at least one predetermined threshold, and the at least one predetermined threshold comprises one or more of: a) a detection of at least 1, 2, 3, 4, 5, 6, 7, 8, 9, or 10 movements by the human subject within 15, 30, 45, 60, 75, or 90 seconds; b) a detection of the human subject, or a portion thereof, moving a distance of at least 5, 6, 7, 8, 9, 10, 11, 12, 13, 14, or 15 cm; c) a velocity of one or more movements by the human subject exceeding 0.1, 0.2, 0.3, 0.4, or 0.5 m/s; d) a degree of rotational movement by the human subject exceeding 1, 2, 3, 4, 5, 6, 7, 8, 9, or 10°; e) a magnitude of one or more accelerations, translational or angular, by the human subject exceeding 0.1, 0.2, 0.3, 0.4, or 0.5 m/s² and/or 90°/s; and/or f) a threshold level of one or more frequencies in a time domain analysis of the data generated by the IMU, wherein fₓ/f₀>5%, where fₓ is a signal strength at a predetermined frequency, optionally wherein the predetermined frequency is 0.25 hz.

In some aspects of the methods described herein, the controller is further configured to cause the stimulation system to stimulate the nerve: a) immediately upon detection of the gross movement; b) after a predetermined amount of time following the detection of the gross movement.

In some aspects of the methods described herein, the controller is further configured to cause the stimulation system to stimulate the nerve: a) for a predetermined amount of time; b) until movement data generated by the IMU indicates that the human subject has a normal respiratory signal; and/or c) until the positional and/or movement data generated by the IMU indicates that the human subject has stopped gross movements.

In some aspects of the methods described herein, stimulating the nerve innervating an upper airway muscle of the human subject comprising stimulating a hypoglossal nerve and/or one or more branches of the hypoglossal nerve, an ansa cervicalis, a vagus nerve, and/or a phrenic nerve of the human subject.

In some aspects of the methods described herein, (a) the IMU is further configured to detect chest and/or abdominal movement by the subject; and (b) the controller is further configured to detect a respiratory signal of the human subject based on the detected chest and/or abdominal movement; and to cause the stimulation system to cease stimulating the nerve or to refrain from stimulating the nerve when the respiratory signal indicates that human subject is not experiencing an apnea or hypopnea event.

In some aspects of the methods described herein, such methods may further comprise detecting a heart rate of the human subject using a heart rate sensor; and one or more parameters of the stimulation of the nerve innervating an upper airway muscle of the human subject are optionally based on the human subject's heart rate, wherein the one or more parameters comprises a start or endpoint time, an intensity level, and/or a duration parameter for stimulation of the nerve.

In some aspects of the methods described herein, the gross movement comprises a heaving motion, and the controller is further configured to detect the heaving motion based on a velocity, acceleration, and/or a direction and/or frequency of the derived positional and/or movement data.

In some aspects of the methods described herein, the gross movement comprises a heaving motion, and the controller is further configured to detect the heaving motion based on a magnitude, a direction, and/or a frequency of the derived positional and/or movement data, within a predetermined time period.

In some aspects of the methods described herein, the controller is further configured to cause stimulation of the nerve after detecting 1, 2, 3, 4, or 5 heaving motions within a predetermined time period.

To the accomplishment of the foregoing and related ends, the one or more aspects comprise the features hereinafter fully described and particularly pointed out in the claims. The following description and the annexed drawings set forth in detail certain illustrative features of the one or more aspects. These features are indicative, however, of but a few of the various ways in which the principles of various aspects may be employed, and this description is intended to include all such aspects and their equivalents.

### BRIEF DESCRIPTION OF THE DRAWINGS

FIG. 1 is a diagram illustrating an exemplary embodiment of a system for treating sleep-disordered breathing using an implantable pulse generator, a cuff electrode on the hypoglossal nerve and an IMU to detect gross movement by the subject being treated.

FIG. 2 is a block diagram illustrating an exemplary embodiment of a system for treating obstructive sleep apnea.

FIG. 3 is a flowchart illustrating aspects of an exemplary method for treating a sleep-disordered breathing condition, such as OSA, using a system according to the present disclosure.

FIG. 4 is a flowchart illustrating aspects of another exemplary method for treating a sleep-disordered breathing condition, such as OSA, using a system according to the present disclosure.

FIG. 5 is a flowchart illustrating aspects of another exemplary method for treating a sleep-disordered breathing condition, such as OSA, using a system according to the present disclosure.

FIG. 6 is a flowchart illustrating aspects of another exemplary method for treating a sleep-disordered breathing condition, such as OSA, using a system according to the present disclosure.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, it will be apparent to those skilled in the art that these concepts may be practiced without these specific details. In some instances, well known structures and components are shown in block diagram form in order to avoid obscuring such concepts.

Several aspects of exemplary embodiments according to the present disclosure will now be presented with reference to various systems and methods. These systems and methods will be described in the following detailed description and illustrated in the accompanying drawings by various blocks, components, circuits, processes, algorithms, etc. (collectively referred to as “elements”). These elements may be implemented using electronic hardware, computer software, or any combination thereof. Whether such elements are implemented as hardware or software depends upon the particular application and design constraints imposed on the overall system.

By way of example, an element, or any portion of an element, or any combination of elements may be implemented as a “processing system” that includes one or more processors. Examples of processors include microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), application-specific integrated circuits (ASICs), state machines, gated logic, discrete hardware circuits, and other suitable hardware configured to perform the various functionality described throughout this disclosure. One or more processors in the processing system may execute software. Software shall be construed broadly to mean instructions, instruction sets, code, code segments, program code, programs, subprograms, software components, applications, software applications, software packages, routines, subroutines, objects, executables, threads of execution, procedures, functions, etc., whether referred to as software, firmware, middleware, microcode, hardware description language, or otherwise.

Accordingly, in one or more exemplary embodiments, the functions described may be implemented in hardware, software, or any combination thereof. If implemented in software, the functions may be stored on or encoded as one or more instructions or code on a computer-readable medium. Computer-readable media includes computer storage media. Storage media may be any available media that can be accessed by a computer. By way of example, and not limitation, such computer-readable media can comprise a random-access memory (RAM), a read-only memory (ROM), an electrically erasable programmable ROM (EEPROM), optical disk storage, magnetic disk storage, other magnetic storage devices, combinations of the aforementioned types of computer-readable media, or any other medium that can be used to store computer executable code in the form of instructions or data structures that can be accessed by a computer.

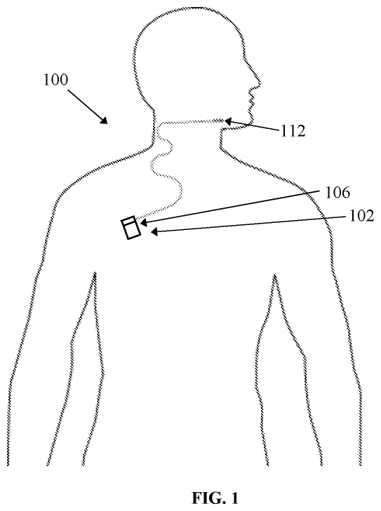

FIG. 1 is a diagram illustrating an embodiment of a system 100 for treating respiratory conditions (e.g., a sleep-disordered breathing condition such as OSA) using an IMU 102. It is envisioned that the IMU 102 may comprise an accelerometer and/or a gyroscope, and be capable of detecting movement by a subject during the inspiration and expiration stages of a respiratory cycle. In some aspects, the accelerometer and/or the gyroscope component may be capable of detecting motion and/or orientation along three axes. The IMU 102 may be configured to generate data based on the detected position and or movement of the human subject. Such positional and/or movement data may comprise, e.g., a signal or data indicative of the degree, magnitude, speed or direction of motion of the subject's chest or any other body part (or one or more portions thereof), and/or orientation data for the subject. In some aspects, absolute or relative timing parameters for any detected motion is derived from positional and/or movement data. In some aspects, an AC component of the IMU 102 data provides chest and/or abdominal movement data and the DC components provide subject orientation data.

In this example, the IMU 102 positioned within the housing containing the stimulation system 106 (e.g., an IPG). The inclusion of an IMU within the housing of an IPG may be advantageous in that it reduces the need for an additional surgical procedure to implant the IMU 102. Prior systems which utilize a pressure sensor to detect movement of the patient's thoracic or abdominal cavity during respiration (a respiration signal) typically require implantation of a sensor in-between a patient's ribs. This requires significant surgical time and tunneling a lead to this space is invasive, resulting in a longer recovery time. Accordingly, the present IMU-based systems are advantageous in that they are able to provide similar respiratory without the need for prolonged surgical and recovery time (i.e., in view of the fact that the IMU 102 can be integrated into the IPG's housing or implanted in proximity thereto in a separate housing). Moreover, as described in further detail below, in alternative aspects the IMU 102 can be positioned outside of the body of the subject (e.g., integrated into a watch, a patch on the chest, or in a housing positioned on the neck, chin, head, or elsewhere).

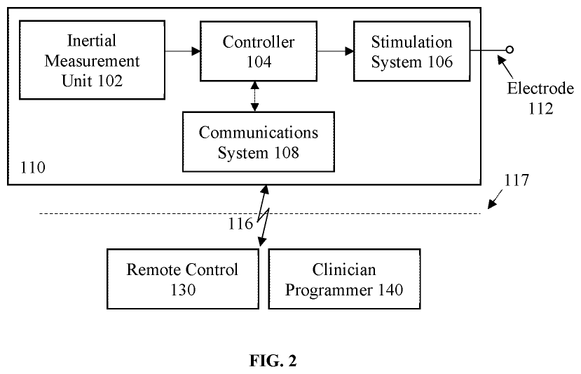

FIG. 2 is a block diagram of an exemplary embodiment of a system 100 for treating obstructive sleep apnea. It includes an implantable stimulator 110, an optional remote control 130, and a clinician programmer 140. In this example, the stimulator 110 includes an IMU 102, a controller 104, a stimulation system 106 (e.g., an IPG), an electrode 112 which may be used to stimulate a respiratory system muscle, and a communications system 108.

The IMU 102 may be configured to detect one or more signals indicative of movement by a subject (e.g., of the chest/abdomen, head, arms, legs, or any other body part or portion thereof) and/or an orientation of the subject, and to generate data based on the detected signals. Positional and/or movement data may comprise, e.g., a signal or data indicative of the degree, magnitude, speed or direction of motion of the subject's chest or any other body part (or one or more portions thereof), and/or orientation data for the subject. The positional and/or movement data can be processed in turn by the controller 104 to detect gross movement(s) by the subject. The stimulation system 106 (e.g., an IPG) may be configured to apply stimulation to the electrode 112. The controller 104 may be configured to control when and/or how the stimulation system 106 applies stimulation to the electrode 112. For example, the controller 104 may be configured to cause stimulation to occur when a gross movement is detected, e.g., immediately, after a predetermined time period (e.g., after 0.5, 1.0, 1.5, 2.0, 2.5, 3.0, 3.5, 4.0, 4.5, or 5.0 seconds, or within a time period defined by any pair of the foregoing values). In some aspects, the controller 104 may be configured to cause stimulation to occur after a predetermined number of gross movements are directed within a predetermined time period (e.g., 1, 2, 3, 4, 5, 6, 7, 8, 9, or 10 gross movements within 0.5, 1.0, 1.5, 2.0, 2.5, 3.0, 3.5, 4.0, 4.5, or 5.0 seconds). In some aspects, the controller 104 may be configured to detect that a gross movement has occurred based on a predetermined thresholds. For example, the controller 104 may be configured to analyze the positional and/or movement data received from the IMU 104 and to detect gross movements based on: a) a number of movements by the human subject within a predetermined time period; b) a linear distance of movement by the human subject; and/or c) a degree of movement along a rotational axis by the human subject.

In some aspects, the controller may be configured to detect the gross movement based on at least one predetermined threshold, e.g., a) a frequency of movements by the human subject; b) a distance of movement by the human subject; c) a velocity and/or acceleration of one or more movements by the human subject; d) a degree of rotational movement by the human subject; e) a magnitude of one or more accelerations, translational or angular, by the human subject; f) a magnitude of one or more velocities, translational or angular, by the human subject; g) an angular acceleration, velocity, and/or number of gross movement events per hour detected by the controller; and/or h) a threshold level of one or more frequencies in a time domain analysis of the IMU data. For example, the controller may be configured to detect gross movement based on: a) a detection of at least 1, 2, 3, 4, 5, 6, 7, 8, 9, or 10 movements by the human subject within 15, 30, 45, 60, 75, or 90 seconds; b) a detection of the human subject, or a portion thereof (e.g., the chest or abdomen), moving a distance of at least 5, 6, 7, 8, 9, 10, 11, 12, 13, 14, or 15 cm; c) a velocity of one or more movements by the human subject exceeding 0.1, 0.2, 0.3, 0.4, or 0.5 m/s; d) a degree of rotational movement by the human subject exceeding 1, 2, 3, 4, 5, 6, 7, 8, 9, or 10°; e) a magnitude of one or more accelerations, translational or angular, by the human subject exceeding 0.1, 0.2, 0.3, 0.4, or 0.5 m/s² and/or 90°/s; and/or f) a threshold level of one or more frequencies in a time domain analysis of the data generated by the IMU, wherein fₓ/f₀>5%, where fₓ is a signal strength at a predetermined frequency, optionally wherein the predetermined frequency is 0.25 hz. It is understood that any number or combination of these thresholds may be used, e.g., a controller may be configured to take into account a degree of rotational movement by the human subject exceeding a predetermined threshold in combination with a detection of the human subject, or a portion thereof (e.g., the chest or abdomen), moving a distance exceeding a second predetermined threshold. Moreover, the detection of gross movement may take into account a frequency of movement (in general or above a threshold acceleration and/or distance) within any time period.

In some aspects, the controller may be configured to cause stimulation to occur until positional and/or movement data from the IMU 102 indicates that the subject has a normal respiratory signal. For example, a detected gross movement may result in application of stimulation throughout an AHI event, and stimulation may be terminated once the positional and/or movement data indicates that the frequency and amplitude of the subject's respiratory cycle are within ranges considered to be normal (e.g., based on a known control, or baseline parameters customized to the subject being treated). In some aspects, a system according to the disclosure may include one or more additional sensors beyond the IMU 102, such as pressure, sound, heart rate, or other sensors, and one or more signals from these sensors may be taken into account by the controller 104 when determining when to apply or cease stimulation, and the parameters of stimulation (e.g., duration and magnitude). For example, an acoustic sensor may be used to detect a respiration signal indicative of the subject's respiratory cycle, and this signal may be used in combination with the positional and/or movement data to determine when to apply or cease stimulation, and/or the parameters of stimulation (e.g., duration and magnitude).

In some aspects, the controller 104 may be configured to temporarily cause constant stimulation for a predetermined time period in response to the detection of one or more gross movements by the subject. The controller 104 may further be configured to adjust the stimulation based on the detection of a respiratory signal for the subject (e.g., using a heart rate, acoustic or other sensor communicatively linked to the controller and capable of detecting a signal indicative of the respiratory cycle of the subject), or a last known good respiratory signal stored in memory.

In some aspects, the controller 104 may be configured to operate in an asynchronous mode wherein a respiratory signal is not used to determine the timing of stimulation. In this asynchronous mode, stimulation may be triggered in response to the detection of gross body movements and applied using a fixed duty cycle.

Systems according to the disclosure may allow the subject being treated to control one or more parameters of the system or of individual components (e.g., the controller 104). For example, the system may allow the subject to place the controller 104 in a standby mode wherein positional and/or movement data is no longer analyzed, where one or more sensors associated with the controller 104 are turned off, and/or where stimulation cannot be triggered. This standby mode may be advantageously turned on when the subject is awake. In some aspects, the subject may similarly be allowed to set a start and/or stop time for monitoring movement (e.g., the subject may wish to set the system to begin monitoring movement at or close to an expected bedtime and to cease monitoring the subject at daybreak). In some aspects, the subject may be allowed to set a time for the system to begin monitoring movement after a specified duration of time (e.g., operation may begin after 10, 15, 20, 25, 30, 35, 40, 45, 50, 55, or 60 minutes), allowing a subject to activate the system at bedtime, so that monitoring and treatment can begin after the subject has gone to sleep.

The controller 104 may further be configured to log the timing, duration, count, and/or severity of AHI events, positional and/or movement data received from the IMU, signals received from one or more communicatively linked sensors, or any other signal or data received by the controller 104. In some aspects, the log may be stored in a local memory (e.g., flash memory) integrated into the controller. Alternatively, the log may be stored in a separate electronic device. For example, the controller may be configured to wirelessly communicate with a computer, smart phone, wearable device, or a dedicated controller and to transmit the log, or a portion thereof, to the separate electronic device in response to user input, input from a clinician programmer, and/or based on a periodic schedule. In some aspects, the controller 104 may be configured to transmit the log, or a portion thereof, when in proximity to the separate electronic device (e.g., when the control 104 detects a signal from a local wireless network and/or a wireless signal associated with the separate electronic device).

In some aspects, the controller 104 may be configured to detect hypopnea events based on the detection of one or more gross movements by the subject. The controller 104 may further be configured to base the detection in part on one or more signals or data received from communicatively linked sensors. For example, hypopnea events may be identified when the controller 104 detects gross movement by the subject (or gross movement above a threshold, optionally within a predetermined time period), plus an increase in hear rate (based on a signal or data received from one or more heart rate sensors communicatively linked to the controller 104). In some aspects, the IMU 102 may be used to detect a heart rate of the subject; in others, a separate sensor may be utilized. The controller 104 may further be configured to cause stimulation of the HGN upon detecting a hypopnea event, and/or to log any such events.

In any of the exemplary aspects described herein, the controller 104 may be configured to apply stimulation continuously, or with a high duty cycle (e.g., >65%) upon detecting (a) gross movement of the subject (or a number of gross movements above a predetermined threshold and/or within a predetermined time period); and/or (b) an apnea or hypopnea event. The start of stimulation may be immediate, or optionally be delayed by a period of time such 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 seconds (or within a range defined by any pair of the foregoing time points). A delay within this range may allow for the subject to potentially recover from the current event and start stimulation before the next event can occur.

In some aspects, the controller 104 may be configured to detect respiratory events (e.g., apnea or hypopnea events) based on the heart rate and respiratory effort of the subject. Apneas and hypopneas cause a restriction in airflow and consequently result in blood oxygen desaturation. Thus, either event may be detected by measuring an increase in heart rate over a short interval of time (e.g., 5, 6, 7, 8, 9 or 10 seconds). For example, an increase of more than 20% average heart rate could be used as a proxy for detecting respiratory events. The controller 104 may be configured to monitor for such events, e.g., by dividing a trailing 10 s heart rate average by a trailing 1 min heard rate. If discrimination between apneas and hypopneas is desired, the controller 104 may be configured, e.g., to detect a greater than 10% increase in average respiratory intensity during the last 10 seconds versus the average respiratory intensity during the last minute. In some aspects, the controller 104 may be configured to adjust a level of stimulation being applied during a respiratory event, based on the detection of gross movement. For example, if an apnea or hypopnea event is detected and stimulation is applied to the HGN, and gross movement is detected, while stimulation is already occurring, this may indicate that the current stimulation level parameters (frequency, amplitude, pulse width, and duty cycle) are insufficient to prevent AHI events, and a new stimulation paradigm could be employed where one or more the stimulation level parameters is changed to increase the likelihood of preventing additional respiratory events. In the alternative, if gross motion is no longer detected, stimulation may also be reduced by adjusting one or more of the stimulation level parameters back towards the original stimulation parameter set, until gross motion is subsequently detected, in which case stimulation may again be increased. A controller 104 configured to implement this model may titrate stimulation for a given subject throughout a given evening/sleep cycle. In some aspects, the controller 104 may be configured to log the final titrated parameter set (e.g., so that it can be used as the starting point for the next evening/sleep cycle).

In some aspects, the controller 104 may be configured to deliver stimulation tailored for a subject having a paradoxical breathing pattern, wherein the chest contracts during inspiration and expands during expiration (i.e., the reverse of a normal breathing pattern). HGNS stimulation is ideally applied at the start of inspiration. Thus, in subjects having a paradoxical breathing pattern, stimulation should be initiated at the start of the contraction phase. Systems according to the disclosure may be configured to operate in a mode tailored for paradoxical breathing (e.g., based upon input provided from the subject or a clinician programmer). In such configurations, the controller 104 may be configured to detect or predict period and amplitude parameters of the subject's respiratory cycle based upon one or more signals or data received from communicatively linked sensors, and to identify the inspiration phase as the phase having a longer period (e.g., inspiration is normally longer than expiration in human subjects, regardless of whether the breathing pattern is normal or paradoxical). In other aspects, the controller 104 may receive a signal or data from a heart rate sensor, and the controller 104 may be configured to identify the inspiration phase based upon the subject's heartbeat. For example, the controller 104 may be configured to use positional and/or movement data from the IMU 102 to identify chest motion indicative of the periods corresponding to inspiration and expiration, and the signal or data from the heart rate sensor to identify the period corresponding to inspiration (a human subject's heart rate is normally faster during inspiration).

The communications system 108 may provide one or more wireless links 116, through the skin 117 of a patient, to a remote control 130, and/or a clinician programmer 140. The remote control 130, and the clinician programmer 140 may also include respective communications systems, which may provide wireless links 116 between the remote control 130, the clinician programmer 140, and/or additional Internet or cloud-based services. Any or all of these wireless links 116 can utilize Bluetooth, Bluetooth Low Energy, or other wireless communication protocols. The wireless links 116 may include authentication and encryption suitable to protect patient data.

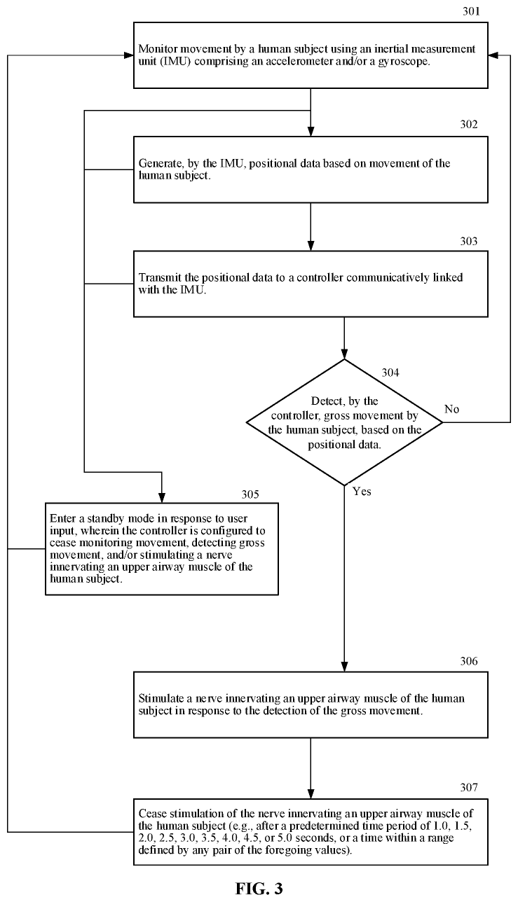

FIGS. 3-6 are flowcharts illustrating several exemplary methods for treating a respiratory condition (e.g., sleep-disordered breathing, or OSA).

As illustrated by FIG. 3, an exemplary method according to the present disclosure may begin with monitoring movement by a human subject using an IMU 102 comprising an accelerometer and/or a gyroscope (301). As explained above, the IMU 102 may be implanted in the human subject (e.g., integrated into the IPG) or external to the human subject (e.g., integrated into a wearable device or placed on the head, chest, neck, arm(s), or leg(s) of the human subject. In some aspects, multiple IMU 102 devices may be used (e.g., one may implanted in the subject and one or more may be provided as external devices that can provide additional positional and/or movement data (e.g., for the human subject, or specific to one or more individual body parts or limbs of the human subject). In either case, the IMU 102 (or plurality thereof) may generate data based on movement of the human subject (302). The IMU 102 (or plurality thereof) may be configured to transmit the positional and/or movement data to a controller 104 communicatively linked to the one or more IMU 102 devices (303). In some aspects, each IMU 102 may be configured to provide real-time positional and/or movement data; in other aspects, each IMU 102 may optionally provide positional and/or movement data periodically or upon receiving a request for updated positional and/or movement data from the controller 104. For example, one IMU 102 may be designated as a primary IMU 102 and provide constant real-time positional and/or movement data to the controller 104, while one or more other IMU 102 devices are placed in a secondary IMU 102 mode wherein positional and/or movement data is only provided periodically (e.g., every 10, 20, 30, 40, 50 or 60 seconds, every 1, 2 3, 4, 5, 6, 7, 8, 9 or 10 minutes, or within a time range with endpoints selected from any of the foregoing time points). Such aspects may be advantageous in that secondary IMU 102 units will use less power and require less processing resources. In some aspects, a controller 104 may be configured to select the best IMU 102 from among a plurality of communicatively linked IMU 102 devices (e.g., based on a signal-to-noise ratio, or any other criterion).

In some aspects, the controller 104 may be configured to detect gross movement by the human subject based on the positional and/or movement data received from one or more IMU 102 devices (304). As explained herein, gross movement may be detected based upon various parameters, including without limitation: a) a number of movements by the human subject (optionally, within a predetermined time period; b) a magnitude of one or more movements by the human subject (optionally, within a predetermined time period); c) a linear distance of movement by the human subject; and/or d) a degree of movement along a rotational axis by the human subject. In some aspects, the controller 104 may be configured to detect a plurality of these movement parameters and to detect gross movement based on a combination of at least two types of movement parameters. In some aspects, the controller 104 may be programmed with one or more thresholds for each of the foregoing movement parameters. For example, the controller 104 may detect gross movement when the IMU 102 detects that the subject's head or chest has moved more than a predefined threshold distance within a defined time period (304). If gross movement is not detected, the process may return to the monitoring stage (301). Alternatively, if gross movement is detected, the controller 104 may be configured to cause stimulation to a neve innervating an upper airway muscle of the human subject (306). The controller may be configured to cease stimulation of the nerve after a predetermined time period (e.g., 1.0, 1.5, 2.0, 2.5, 3.0, 3.5, 4.0, 4.5, or 5.0 seconds. As illustrated by this figure, at any point in this workflow, the controller 104 may be configured to enter a standby mode in which the controller 104 is configured to cease monitoring movement, detecting gross movement, and/or stimulating the nerve (305). This standby mode may be selected by the human subject, by a clinician programmer, or based on a programmed schedule, and may be particularly advantageous when a user is awake or otherwise does not require stimulation. The controller 104 may exit standby mode based upon further input from the human subject or clinician programmer, or based upon a schedule.

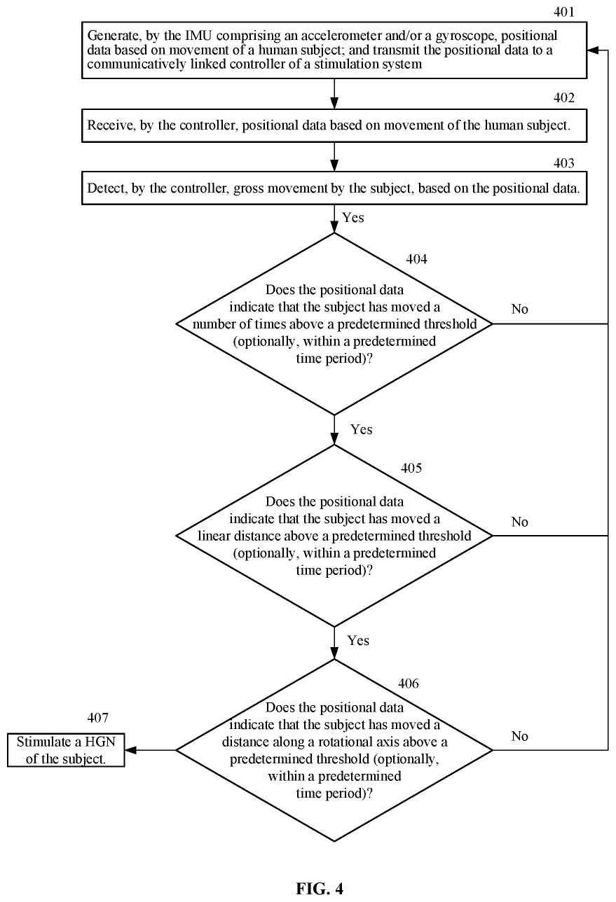

FIG. 4 illustrates another example of a method for treating a respiratory condition (e.g., sleep-disordered breathing or OSA). In this example, the process begins with an IMU 102 generating positional and/or movement data based on movement of the human subject, wherein the IMU 102 comprises an accelerometer and/or a gyroscope. The positional and/or movement data is then transmitted to a communicatively linked controller 104 (401). Upon receiving the positional and/or movement data (402), the controller may then detect gross movement by the human subject using the positional and/or movement data (403).

In this example, it is shown that the controller 104 may first detect whether the positional and/or movement data indicates that the subject has moved a number of times above a predetermined threshold (optionally, within a predetermined time period) (404); then whether the positional and/or movement data indicates that the subject has moved a linear distance above a predetermined threshold (optionally, within a predetermined time period) (405); and finally whether the positional and/or movement data indicates that the subject has moved a distance along a rotational axis above a predetermined threshold (optionally, within a predetermined time period) (406). In this example, if all three types of movement are detected and pass the required thresholds, stimulation is applied to the HGN (407). However, this figure depicts a non-limiting example. In other aspects, a system according to the disclosure may perform any of the movement analyses (steps 404-406), in any order or in parallel. Indeed, gross movement may be detected based upon a finding that one of these parameters is satisfied, or any combination of the three. In other aspects, other parameters may be evaluated as part of (or in place of) the exemplary parameters analyzed in steps 404-406.

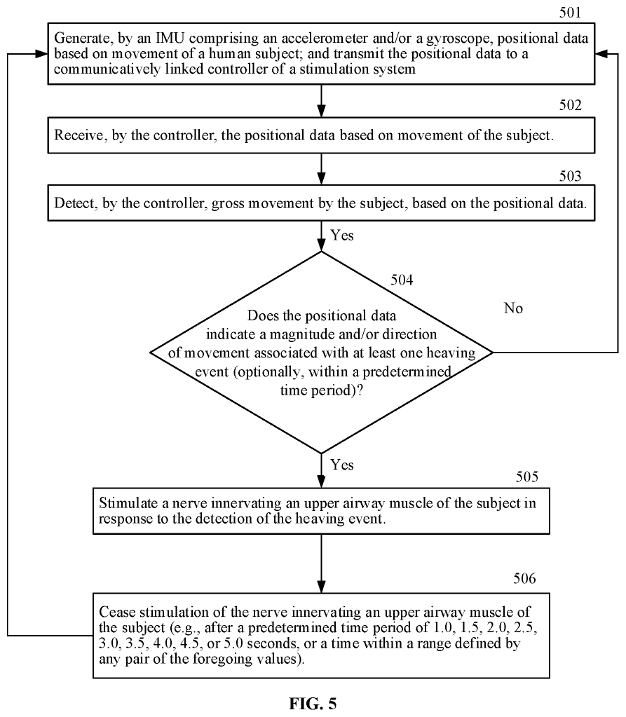

FIG. 5 illustrates another exemplary method of treatment according to the disclosure. This aspect is similar to the one shown in FIG. 4 (e.g., steps 501-503). In this case, the controller 104 is configured to take into account a direction and/or magnitude of movement by the human subject (e.g., an implanted or external IMU 102 may provide positional and/or movement data indicating that the subject's chest quickly and forcefully moved in a direction indicative of a heaving motion). Upon detection of a heaving event (501), the controller 104 is configured to cause stimulation of the at least on nerve innervating an upper airway muscle of the subject (e.g., the HGN) (505). Next, the controller 104 is configured to cease stimulation after a predetermined time period (506).

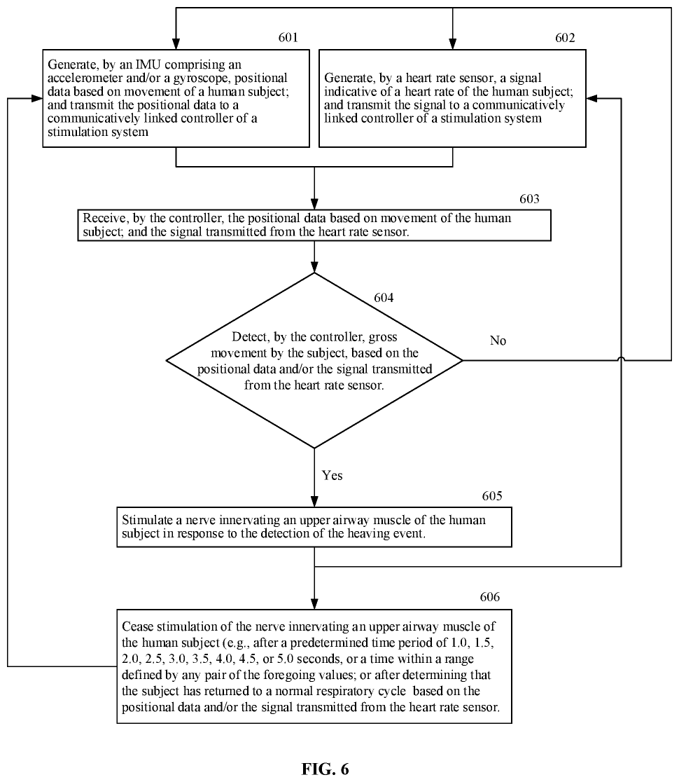

FIG. 6 exemplifies another potential method according to the disclosure. In this case, a heart rate sensor 114 is used to collect additional data regarding the human subject. As noted above, in some aspects an IMU 104 may provide a signal indicative of a heart rate of the human subject (e.g., an IMU 104 implanted in the chest of the subject may be capable of detecting a heart rate signal). Here, the heart rate sensor 114 is presumed to be integrated into a housing configured to be worn by the user (e.g., in a wearable device). Thus, the method begins with the generation of positional and/or movement data based on movement of the subject, using the IMU 102 (601). In sequence, or in parallel as shown here, a heart rate sensor 114 is configured to detect a signal indicative of a heart rate of the subject (602). Both devices are configured to transmit sensor data to a communicatively linked controller 104. Upon receiving the positional and/or movement data and the heart rate data (603), the controller 104 shall then detect gross movement(s) by the subject based on the positional and/or movement data and/or the signal transmitted from the heart rate sensor (604). If gross movement is not detected, the system may return to the initial monitoring steps (601, 602). Alternatively, if gross movement is detected, the controller 104 may be configured to cause stimulation of a nerve innervating an upper airway muscle of the subject (605). Finally, at step 606, the controller may cease stimulation (e.g., after a predetermined time period, or after determining that the subject has returned to a normal respiratory cycle). For the respiratory cycle comparison, parameters from a baseline for the human subject, or a known control, may be used.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular compound, composition, article, apparatus, methodology, protocol, and/or reagent, etc., described herein, unless expressly stated as such. In addition, those of ordinary skill in the art will recognize that certain changes, modifications, permutations, alterations, additions, subtractions and sub-combinations thereof can be made in accordance with the teachings herein without departing from the spirit of the present specification. It is therefore intended that the following appended claims and claims hereafter introduced are interpreted to include all such changes, modifications, permutations, alterations, additions, subtractions and sub-combinations as are within their true spirit and scope.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

Groupings of alternative embodiments, elements, or steps of the present invention are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques.

Use of the terms “may” or “can” in reference to an embodiment or aspect of an embodiment also carries with it the alternative meaning of “may not” or “cannot.” As such, if the present specification discloses that an embodiment or an aspect of an embodiment may be or can be included as part of the inventive subject matter, then the negative limitation or exclusionary proviso is also explicitly meant, meaning that an embodiment or an aspect of an embodiment may not be or cannot be included as part of the inventive subject matter. In a similar manner, use of the term “optionally” in reference to an embodiment or aspect of an embodiment means that such embodiment or aspect of the embodiment may be included as part of the inventive subject matter or may not be included as part of the inventive subject matter. Whether such a negative limitation or exclusionary proviso applies will be based on whether the negative limitation or exclusionary proviso is recited in the claimed subject matter.

Notwithstanding that the numerical ranges and values setting forth the broad scope of the invention are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar references used in the context of describing the present invention (especially in the context of the following claims) are to be construed to cover both the singular and the plural, unless otherwise indicated herein or clearly contradicted by context. Further, ordinal indicators—such as “first,” “second,” “third,” etc.—for identified elements are used to distinguish between the elements, and do not indicate or imply a required or limited number of such elements, and do not indicate a particular position or order of such elements unless otherwise specifically stated. All methods described herein can be performed in any suitable order unless otherwise indicated herein or otherwise clearly contradicted by context. The use of any and all examples, or exemplary language (e.g., “such as”) provided herein is intended merely to better illuminate the present invention and does not pose a limitation on the scope of the invention otherwise claimed. No language in the present specification should be construed as indicating any non-claimed element essential to the practice of the invention.

When used in the claims, whether as filed or added per amendment, the open-ended transitional term “comprising” (and equivalent open-ended transitional phrases thereof like including, containing and having) encompasses all the expressly recited elements, limitations, steps and/or features alone or in combination with unrecited subject matter; the named elements, limitations and/or features are essential, but other unnamed elements, limitations and/or features may be added and still form a construct within the scope of the claim. Specific embodiments disclosed herein may be further limited in the claims using the closed-ended transitional phrases “consisting of” or “consisting essentially of” in lieu of or as an amended for “comprising.” When used in the claims, whether as filed or added per amendment, the closed-ended transitional phrase “consisting of” excludes any element, limitation, step, or feature not expressly recited in the claims. The closed-ended transitional phrase “consisting essentially of” limits the scope of a claim to the expressly recited elements, limitations, steps and/or features and any other elements, limitations, steps and/or features that do not materially affect the basic and novel characteristic(s) of the claimed subject matter. Thus, the meaning of the open-ended transitional phrase “comprising” is being defined as encompassing all the specifically recited elements, limitations, steps and/or features as well as any optional, additional unspecified ones. The meaning of the closed-ended transitional phrase “consisting of” is being defined as only including those elements, limitations, steps and/or features specifically recited in the claim whereas the meaning of the closed-ended transitional phrase “consisting essentially of” is being defined as only including those elements, limitations, steps and/or features specifically recited in the claim and those elements, limitations, steps and/or features that do not materially affect the basic and novel characteristic(s) of the claimed subject matter. Therefore, the open-ended transitional phrase “comprising” (and equivalent open-ended transitional phrases thereof) includes within its meaning, as a limiting case, claimed subject matter specified by the closed-ended transitional phrases “consisting of” or “consisting essentially of.” As such embodiments described herein or so claimed with the phrase “comprising” are expressly or inherently unambiguously described, enabled and supported herein for the phrases “consisting essentially of” and “consisting of.”

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Lastly, the terminology used herein is for the purpose of describing particular embodiments only, and is not intended to limit the scope of the present invention, which is defined solely by the claims. Accordingly, the present invention is not limited to that precisely as shown and described.

What is claimed is:

1. A system for treating a respiratory disorder, comprising:
an inertial measurement unit (IMU) comprising an accelerometer and/or a gyroscope, wherein the IMU is configured to detect movement by a human subject, and to generate data based on the detected movement; and
a stimulator comprising:
a stimulation system configured to deliver stimulation to a nerve which innervates an upper airway muscle; and
a controller coupled to the stimulation system, and to the IMU;
wherein the controller is configured to detect gross movement by the human subject based on the data generated by the IMU, and to cause the stimulation system to stimulate the nerve in response to the detection of the gross movement.

2. The system of claim 1, wherein the controller is further configured to detect the gross movement based on at least one predetermined threshold.

3. The system of claim 2, wherein the at least one predetermined threshold comprises:
a) a frequency of movements by the human subject;
b) a distance of movement by the human subject;
c) a velocity and/or acceleration of one or more movements by the human subject;
d) a degree of rotational movement by the human subject;
e) a magnitude of one or more accelerations, translational or angular, by the human subject;
f) a magnitude of one or more velocities, translational or angular, by the human subject;
g) an angular acceleration, velocity, and/or number of gross movement events per hour detected by the controller; and/or
h) a threshold level of one or more frequencies in a time domain analysis of the IMU data.

4. The system of claim 2, wherein the controller is further configured to detect the gross movement based on at least one predetermined threshold, and the at least one predetermined threshold comprises one or more of:
a) a detection of at least 1, 2, 3, 4, 5, 6, 7, 8, 9, or 10 movements by the human subject within 15, 30, 45, 60, 75, or 90 seconds;
b) a detection of the human subject, or a portion thereof, moving a distance of at least 5, 6, 7, 8, 9, 10, 11, 12, 13, 14, or 15 cm;
c) a velocity of one or more movements by the human subject exceeding 0.1, 0.2, 0.3, 0.4, or 0.5 m/s;
d) a degree of rotational movement by the human subject exceeding 1, 2, 3, 4, 5, 6, 7, 8, 9, or 10°;
e) a magnitude of one or more accelerations, translational or angular, by the human subject exceeding 0.1, 0.2, 0.3, 0.4, or 0.5 m/s² and/or 90°/s; and/or
f) a threshold level of one or more frequencies in a time domain analysis of the data generated by the IMU, wherein fₓ/f₀>5%, where fₓ is a signal strength at a predetermined frequency, optionally wherein the predetermined frequency is 0.25 hz.

5. The system of claim 1, wherein the controller is further configured to cause the stimulation system to stimulate the nerve:
a) immediately upon detection of the gross movement; or
b) after a predetermined amount of time following the detection of the gross movement.

6. The system of claim 1, wherein the controller is further configured to cause the stimulation system to stimulate the nerve:
a) for a predetermined amount of time;
b) until the data generated by the IMU indicates that the human subject has a normal respiratory signal; or
c) until the data generated by the IMU indicates that the human subject has stopped gross movements.

7. The system of claim 1, wherein the stimulation system is configured to deliver stimulation to a hypoglossal nerve and/or one or more branches of the hypoglossal nerve, and/or an ansa cervicalis, and/or a vagus nerve, and/or a phrenic nerve of the human subject.

8. The system of claim 1, wherein the IMU is further configured to detect chest and/or abdominal movement by the subject; and
the controller is further configured to detect a respiratory signal of the human subject based on the detected chest and/or abdominal movement; and to cause the stimulation system to cease stimulating the nerve or to refrain from stimulating the nerve, when the respiratory signal indicates that human subject is not experiencing an apnea or hypopnea event.

9. The system of claim 1, wherein the system further comprises a heart rate sensor; and the controller is further configured to detect a heart rate of the human subject, and to take the human subject's heart rate into account when selecting a start or endpoint time, an intensity level, and/or a duration parameter for stimulation of the nerve.

10. The system of claim 1, wherein the system is further configured to receive input from the human subject allowing a clinician or the human subject to control one or more parameters of the stimulation system.

11. The system of claim 10, wherein the controller is further configured to allow the clinician or the human subject to place the controller in a standby mode in which the controller ceases to detect gross movement by the human subject and/or cause stimulation of the nerve.

12. The system of claim 1, wherein the gross movement comprises a heaving motion of a chest and/or abdomen of the human subject, and the controller is further configured to detect the heaving motion based on a velocity, and/or an acceleration, and/or a direction, and/or a frequency of the heaving motion calculated based on the data generated by the IMU.

13. The system of claim 1, wherein the gross movement comprises a heaving motion, and the controller is further configured to detect the heaving motion based on a magnitude, direction, and/or frequency of the heaving motion calculated using the data generated by the IMU.

14. The system of claim 12, wherein the controller is further configured to cause stimulation of the nerve based on a frequency of the heaving motions; optionally, after detecting 1, 2, 3, 4, or 5 heaving motions within a predetermined time period.

15. A method for treating sleep-disordered breathing, comprising:
detecting movement by a human subject using an inertial measurement unit (IMU) comprising an accelerometer and/or a gyroscope;
deriving positional and/or movement data based on the IMU data;
detecting, by a controller communicatively linked with the IMU, gross movement by the human subject based on the positional and/or movement data generated by the IMU; and
stimulating a nerve innervating an upper airway muscle of the human subject in response to the detection of the gross movement.

16. The method of claim 15, wherein the controller is further configured to detect the gross movement based on at least one predetermined threshold.

17. The method of claim 15, wherein the at least one predetermined threshold comprises:
a) a frequency of movements by the human subject;
b) a distance of movement by the human subject;
c) a velocity and/or acceleration of one or more movements by the human subject;
d) a degree of rotational movement by the human subject;
e) a magnitude of one or more accelerations, translational or angular, by the human subject;
f) a magnitude of one or more velocities, translational or angular, by the human subject;
g) an angular acceleration, velocity, and/or number of gross movement events per hour detected by the controller; and/or
h) a threshold level of one or more frequencies in a time domain analysis of the IMU data.

18. The method of claim 15, wherein the controller is further configured to detect the gross movement based on at least one predetermined threshold, and the at least one predetermined threshold comprises one or more of:
a) a detection of at least 1, 2, 3, 4, 5, 6, 7, 8, 9, or 10 movements by the human subject within 15, 30, 45, 60, 75, or 90 seconds;
b) a detection of the human subject, or a portion thereof, moving a distance of at least 5, 6, 7, 8, 9, 10, 11, 12, 13, 14, or 15 cm;
c) a velocity of one or more movements by the human subject exceeding 0.1, 0.2, 0.3, 0.4, or 0.5 m/s;
d) a degree of rotational movement by the human subject exceeding 1, 2, 3, 4, 5, 6, 7, 8, 9, or 10°;
e) a magnitude of one or more accelerations, translational or angular, by the human subject exceeding 0.1, 0.2, 0.3, 0.4, or 0.5 m/s² and/or 90°/s; and/or
f) a threshold level of one or more frequencies in a time domain analysis of the data generated by the IMU, wherein fₓ/f₀>5%, where fₓ is a signal strength at a predetermined frequency, optionally wherein the predetermined frequency is 0.25 hz.

19. The method of claim 15, wherein the controller is further configured to cause the stimulation system to stimulate the nerve:
a) immediately upon detection of the gross movement;
b) after a predetermined amount of time following the detection of the gross movement.

20. The method of claim 15, wherein the controller is further configured to cause the stimulation system to stimulate the nerve:
a) for a predetermined amount of time;
b) until movement data generated by the IMU indicates that the human subject has a normal respiratory signal; and/or
c) until the positional and/or movement data generated by the IMU indicates that the human subject has stopped gross movements.

21. The method of claim 15, wherein stimulating the nerve innervating an upper airway muscle of the human subject comprising stimulating a hypoglossal nerve and/or one or more branches of the hypoglossal nerve, an ansa cervicalis, a vagus nerve, and/or a phrenic nerve of the human subject.

22. The method of claim 15, wherein the IMU is further configured to detect chest and/or abdominal movement by the subject; and
the controller is further configured to detect a respiratory signal of the human subject based on the detected chest and/or abdominal movement; and to cause the stimulation system to cease stimulating the nerve or to refrain from stimulating the nerve when the respiratory signal indicates that human subject is not experiencing an apnea or hypopnea event.

23. The method of claim 15, further comprising:
detecting a heart rate of the human subject using a heart rate sensor; and
one or more parameters of the stimulation of the nerve innervating an upper airway muscle of the human subject are optionally based on the human subject's heart rate,
wherein the one or more parameters comprises a start or endpoint time, an intensity level, and/or a duration parameter for stimulation of the nerve.

24. The method of claim 15, wherein the gross movement comprises a heaving motion, and the controller is further configured to detect the heaving motion based on a velocity, acceleration, and/or a direction and/or frequency of the derived positional and/or movement data.

25. The method of claim 24, wherein the gross movement comprises a heaving motion, and the controller is further configured to detect the heaving motion based on a magnitude, a direction, and/or a frequency of the derived positional and/or movement data, within a predetermined time period.

26. The method of claim 25, wherein the controller is further configured to cause stimulation of the nerve after detecting 1, 2, 3, 4, or 5 heaving motions within a predetermined time period.
